(19) United States
(12) Patent Application Publication
(10) Pub. No.: US 2026/0131125 A1
(43) Pub. Date: May 14, 2026

(54) VALVE ASSEMBLY FOR AN INTRODUCER SHEATH

(71) Applicant: W. L. Gore & Associates, Inc., Newark, DE (US)

(72) Inventors: David J. Arcaro, Flagstaff, AZ (US); Kyle R. Buckley, Flagstaff, AZ (US); Hey Sang Kim, Phoenix, AZ (US)

(21) Appl. No.: 19/385,324

(22) Filed: Nov. 11, 2025

Related U.S. Application Data
(60) Provisional application No. 63/719,377, filed on Nov. 12, 2024.

(51) Int. Cl. A61M 39/06; A61M 25/06

(52) U.S. Cl. CPC A61M 39/0613; A61M 25/0662; A61M 2025/0681; A61M 2039/062

(57) ABSTRACT

A valve assembly comprises an outer tube, an inner tube arranged at least partially within the outer tube to define a pressurizable space therebetween, the inner tube extending along a longitudinal axis between a first end and a second end and formed of a drapeable material, the inner tube having a center portion intermediate the first end and the second end, a first end ring positioned adjacent the first end, the first end ring including a plurality of axially adjacent portions, the plurality of axially adjacent portions comprising a first portion formed of a first material, and a second portion positioned axially adjacent the first portion and formed of a second material.

### CROSS-REFERENCE TO RELATED APPLICATION

This application claims the benefit of Provisional Application No. 63/719,377, filed Nov. 12, 2024, which is incorporated herein by reference in its entirety for all purposes.

### BACKGROUND

During endoluminal procedures, an insertion site is formed through a patient's skin to gain access to the vasculature of the patient. These insertion sites also provide a path for blood to flow from the patient that needs to be controlled (sealed). Generally, endoluminal procedures utilize introducer sheaths, or introducer sheath assemblies, with some type of hemostasis valve for reducing or preventing blood flow through the introducer sheath, including reducing or preventing blood flow past an endoprosthesis system introduced into the vasculature through the introducer sheath.

### SUMMARY

Various examples addressed in this patent specification relate to introducer sheath valve designs that facilitate one or more of: ease of manufacture, enhanced efficacy, and/or improved reliability. For example, various concepts relate to a valve assembly for an introducer sheath, the valve assembly including an inner tube through which an endoluminal system (e.g., a transcatheter delivery system) may be passed, with the inner tube being able to seal against the endoluminal system. More specifically, various concepts relate to coupling / seal designs for the inner tube that facilitate securing the inner tube within the valve assembly.

For example, some introducer sheath assembly embodiments include a pair of flexible members (e.g., an inner tube and an outer tube arranged coaxially) that are radially separated to create a pressurizable space. In order to effectively seal and couple the tubes relative to one another (e.g., through attachment to an associated housing), the one or more flexible members may include one or more retention features to secure the flexible member(s) and thereby help maintain an operative seal for the pressurizable space. For example, retention features may be formed on tube members by coupling, adhesion, printing, or other methods to maintain strength and cohesion of the flexible members. The retention features may be in the form of rings, annuluses, or other circumferentially extending features, for example.

The valve description and examples are generally provided in the context of a hemostasis, or hemostatic valve, such as those used in association with an introducer sheath. The valve designs and concepts provided in this patent specification can also be applied to other types of valves, and a variety of applications, and thus the scope of the inventive concepts addressed herein should be read broadly, and to be applicable to a variety of functional valve types.

According to one example (“Example 1”), a valve assembly comprises an outer tube, an inner tube arranged at least partially within the outer tube to define a pressurizable space therebetween, the inner tube extending along a longitudinal axis between a first end and a second end and formed of a drapeable material, the inner tube having a center portion intermediate the first end and the second end, a first end ring positioned adjacent the first end, the first end ring including a plurality of axially adjacent portions, the plurality of axially adjacent portions comprising a first portion formed of a first material, and a second portion positioned axially adjacent the first portion and formed of a second material.

According to another example (“Example 2”), further to Example 1, the first portion is longitudinally offset from the second portion.

According to another example (“Example 3”), further to any of the preceding Examples, the inner tube is formed of a third material and the second material has a lower melting point than the third material.

According to another example (“Example 4”), further to Example 3, the third material is of the same class of material as the first material.

According to another example (“Example 5”), further to any of the preceding Examples, the second material has a melting temperature less than or equal to 130 degrees Celsius.

According to another example (“Example 6”), further to any of the preceding Examples, the first end ring is coupled to the inner tube by melting the first layer with the inner tube.

According to another example (“Example 7”), further to any of the preceding Examples, the first end ring includes a keyed portion.

According to another example (“Example 8”), further to Example 7, a first collar is coupled to the outer tube and the inner tube such that the first collar interfaces with the keyed portion of the first end ring.

According to another example (“Example 9”), further to any of the preceding Examples, the inner tube is formed of an expanded polyethylene (ePE).

According to another example (“Example 10”), further to any of Examples 1-8, the inner tube is formed of an Aliphatic Thermoplastic Polyether Polyurethane (ATPU).

According to one example (“Example 11”), an introducer sheath assembly comprises an outer tube, an inner tube arranged at least partially within the outer tube to create a pressurizable space between the outer tube and the inner tube, the inner tube formed of a drapeable material and comprising a first end, a second end, a first end ring adjacent the first end, the first end ring comprising a first ring portion formed of a first material and a second ring portion formed of a second material and coupled to the first ring portion.

According to one example (“Example 12”), according to Example 11, a fitting is coupled to the outer tube relative to the inner tube to define the pressurizable space, the fitting operable to engage the inner tube at the first end ring.

According to another example (“Example 13”), further to any of Examples 11-12, a second end ring is adjacent the second end, the second end ring comprising a first ring portion formed of the second material and a second ring portion coupled to the first ring portion.

According to another example (“Example 14”), further to Example 13, a collar is operable to engage the second end ring.

According to another example (“Example 15”), further to any of Examples 11-14, the inner tube is formed of a third material, and the second material has a lower melting point than the third material.

According to another example (“Example 16”), further to Example 15, the third material is the same as the first material.

According to another example (“Example 17”), further to any of Examples 11-16, the first ring portion and the second ring portion are axially offset.

According to another example (“Example 18”), further to any of Examples 11-17, the outer tube is formed of a flexible material such that upon pressurizing the pressurizable space, the outer tube distends outwardly.

According to another example (“Example 19”), further to any of Examples 11-18, the inner tube is formed of a flexible material such that upon pressurizing the pressurizable space, the inner tube distends inwardly.

According to another example (“Example 20”), further to any of Examples 11-19, the inner tube is formed of an expanded polyethylene (ePE).

According to another example (“Example 21”), further to any of Examples 11-19, the inner tube is formed of an Aliphatic Thermoplastic Polyether Polyurethane (ATPU).

According to one example (“Example 22”), a method of utilizing an introducer sheath assembly including an outer tube, an inner tube formed of a drapeable material, the inner tube arranged within the outer tube and including a first end, a second end, a first end ring adjacent the first end ring and the second end ring adjacent the second end ring, a center portion between the first end ring and the second end ring, a pressurizable space defined between the inner tube and the outer tube, the first end ring comprising a first ring portion formed of a second material and a second ring portion formed of a third material, the first ring portion coupled to the second ring portion, a first collar coupled to the outer tube relative to the inner tube such that the first end ring is engaged by the first collar, and a fill port fluidly coupled to the pressurizable space, the method comprising providing a tool insertable into the inner tube, pressurizing the pressurizable space with a fluid input to the fill port, and reducing a diameter of the center portion in response to the pressurizing such that the center portion sealingly collapses around the tool.

According to another example (“Example 23”), further to Example 22, the outer tube is formed of a flexible material, the method further comprising distending the outer tube in response to the pressurizing.

According to another example (“Example 24”), further to any of Examples 22-23, the second end ring comprises a first ring portion formed of the second material and a second ring portion formed of the third material.

According to another example (“Example 25”), further to any of Examples 22-24, a second collar is coupled to the outer tube relative to the inner tube such that the second end ring is engaged by the second collar.

According to another example (“Example 26”), further to any of Examples 22-25, the second material has a lower melting point than the third material.

According to another example (“Example 27”), further to Example 26, the first ring portion with the second material is adhered with the first material to couple the first ring portion to the inner tube.

According to another example (“Example 28”), further to any of Examples 22-27, the inner tube is formed of an expanded polyethylene (ePE).

According to another example (“Example 29”), further to any of Examples 22-27, the inner tube is formed of an Aliphatic Thermopalstic Polyether Polyurethane (ATPU).

According to one example (“Example 30”), a method of manufacturing a medical device comprises forming an inner tube formed of a drapeable material and defining a first end portion, a second end portion, and a center portion, wherein the inner tube is formed of a first material, forming a first end ring defining a first ring portion formed of a second material and a second ring portion coupled to the first ring portion, the second ring portion formed of a third material different from the second material, positioning the first end ring proximate the first end portion of the inner tube, and coupling the first end ring to the inner tube by adhering the first ring portion to the inner tube.

According to another example (“Example 31”), further to Example 30, the method further comprising forming a second end ring defining a first ring portion formed of the second material and a second ring portion coupled to the first ring portion, the second ring portion formed of the third material.

According to another example (“Example 32”), further to any of Examples 30-31, the second material has a melting temperature less than the third material.

According to another example (“Example 33”), further to any of Examples 30-32, the third material is of the same class of material as the first material, and the second material has a melting temperature less than or equal to 130 degrees Celsius.

According to another example (“Example 34”), further to any of Examples 30-33, the first ring portion surrounds the second ring portion.

According to another example (“Example 35”), further to any of Examples 30-34, the inner tube is formed of an expanded polyethylene (ePE).

According to another example (“Example 36”), further to any of Examples 30-34, the inner tube is formed of an Aliphatic Thermoplastic Polyether polyurethane (ATPU).

According to one example (“Example 37”), a method for making an introducer sheath assembly comprises forming an inner tube formed of a drapeable material and defining a first end portion, a second end portion, and a center portion intermediate the first end portion and the second end portion, wherein the inner tube is formed of a first material, forming a first end ring defining a first end ring portion and a second end ring portion coupled to the first end ring portion, the second end ring portion formed of the first material, coupling the first end ring to the inner tube adjacent the first end portion, positioning the inner tube within an outer tube to create a pressurizable space therebetween, and coupling a first collar to each of the outer tube and the inner tube such that the first end ring is operable to constrain the inner tube relative to the outer tube.

According to another example (“Example 38”), further to Example 37, further comprising forming a second end ring defining a first ring portion and a second ring portion coupled to the first ring portion, the second ring portion formed of the first material, and coupling the second end ring to the inner tube adjacent the second end portion.

According to another example (“Example 39”), further to Example 38, further comprising coupling a second collar to each of the outer tube and the inner tube such that the second end ring is operable to engage the inner tube relative to the outer tube.

According to another example (“Example 40”), further to any of Examples 37-39, the first ring portion is formed of a second material, and the first material has a lower melting temperature than the second material.

According to another example (“Example 41”), further to Example 40, the inner tube extends along an axis, and the first ring portion is axially offset from the second ring portion.

According to another example (“Example 42”), further to any of Examples 37-41, the inner tube is formed of an expanded polyethylene (ePE).

According to another example (“Example 43”), further to any of Examples 37-41, the inner tube is formed of an Aliphatic Thermoplastic Polyether Polyurethane (ATPU).

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is a perspective view of an introducer sheath in accordance with an embodiment;

FIG. 2 is a perspective view of the introducer sheath of FIG. 1;

FIG. 3 is an exploded view of the introducer sheath of FIG. 1;

FIG. 4 is a section view of the introducer sheath of FIG. 1;

FIG. 5 is an end on view of the introducer sheath of FIG. 1;

FIG. 6 is a perspective view of an end ring of an introducer sheath in accordance with an embodiment;

FIG. 7 is a side view of the end ring of FIG. 6;

FIG. 8 is an exploded side view of the end ring of FIG. 6;

FIG. 9 is a side view of a tube on a mandrel showing end rings positioned off of the tube in accordance with an embodiment;

FIG. 10 is a side view of the tube of FIG. 9 showing the end rings positioned on the tube;

FIG. 11 is a perspective view of the tube of FIG. 10;

FIG. 12A is a perspective view an end ring in accordance with an embodiment;

FIG. 12B is a side view of the end ring of FIG. 12A;

FIG. 13A is a perspective view of an end ring in accordance with an embodiment;

FIG. 13B is a side view of the end ring of FIG. 13A;

FIG. 14 is a side view of a tube on a mandrel adjacent an extruder in accordance with an embodiment;

FIG. 15 is a side view of the tube of FIG. 14 showing the extruder creating an end ring as well as an optional second extruder creating a second end ring in accordance with an embodiment;

FIG. 16 is a side view of the tube of FIG. 14 showing a completed end ring;

FIG. 17 is a section view of the tube of FIG. 16 taken along section 17-17 of FIG. 16;

FIG. 18 is a section view of the extruder and tube of FIG. 16 taken along line 18-18 of FIG. 16;

FIG. 19 is a partially exploded view of an introducer sheath assembly of the present disclosure in accordance with an embodiment;

FIG. 20 is an exploded view of the introducer sheath assembly of FIG. 19;

FIG. 21 is a section view of the introducer sheath assembly of FIG. 19 in an unpressurized state, taken along a horizontal plane extending along the longitudinal axis of the introducer sheath assembly of FIG. 19;

FIG. 22 is a section view of the introducer sheath assembly of FIG. 19 in a pressurized state, taken along a horizontal plane extending along the longitudinal axis of the introducer sheath assembly of FIG. 19;

FIG. 23 is a front view of a molding assembly with a separated from a tube member in accordance with an embodiment;

FIG. 24 is a front view of a molding assembly surrounding a tube member in accordance with an embodiment;

FIG. 25 is a perspective view of a molding assembly separated apart in accordance with an embodiment;

FIG. 26 is a perspective view of a molding assembly separated from a tube member in accordance with an embodiment;

FIG. 27 is a side view of a tube member in accordance with an embodiment;

FIG. 28 is a front view of a tube member in accordance with an embodiment; and

FIG. 29 is a front view of a tube member in accordance with an embodiment.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

The term “drape” or “drapeable” can be understood to mean the way a fabric or a material falls when hung in different positions such as the ability of the fabric or material to conform to the shape of another object when laid upon it. Drape is associated with flexibility and suppleness of a fabric or a material.

### Description of Various Embodiments

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

Various embodiments addressed in this patent specification relate to valve assembly coupling and sealing features, and more specifically valve assemblies usable in introducer sheaths, for example. The concepts addressed include enhanced manufacturability, efficacy, and/or reliability in association with various components, and particular in association with the inner sealing member, or sealing tube that can conform to device passed through the valve assembly to seal around the valve assembly. Various embodiments include assembling end rings with an inner tube of the valve assembly by various methods including melting, additive manufacturing, various forms of welding, and other methods.

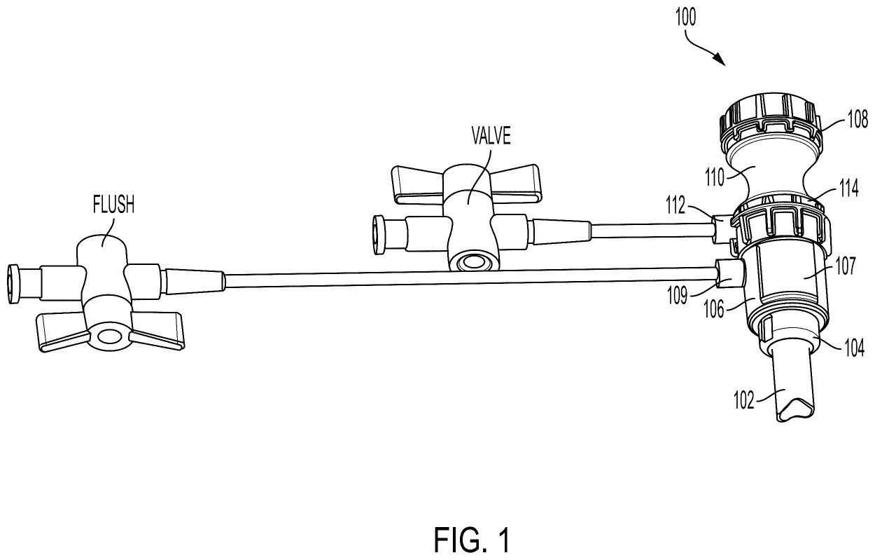

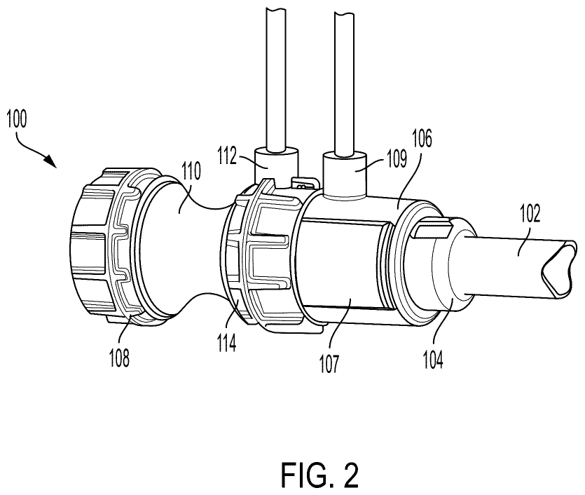

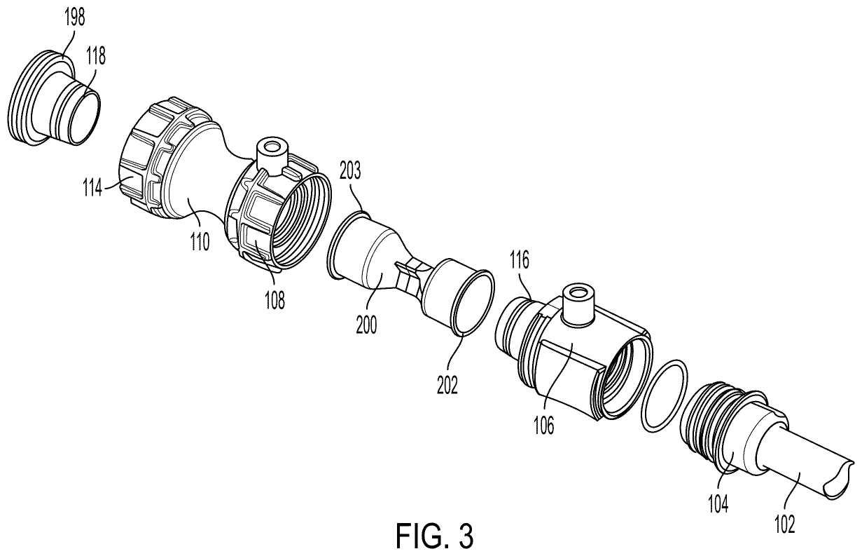

The introducer sheath assembly shown in FIG. 1 is provided as an example of the various features of the introducer sheath assembly and, although the combination of those illustrated features is clearly within the scope of invention, that example and its illustration is not meant to suggest the inventive concepts provided herein are limited from fewer features, additional features, or alternative features to one or more of those features shown in FIG. 1. For example, as another example, the introducer sheath assembly shown in FIG. 1 may include the inner tube described with reference to FIG. 10. It should also be understood that the reverse is true as well. One or more of the components depicted in FIG. 1 can be employed in addition to, or as an alternative to components depicted in FIG. 10. For example, the end rings of the inner tube shown in FIG. 3 may be employed in connection with the inner tube shown in FIG. 10.

As described in greater detail, the introducer sheath assembly of FIG. 1 may include end ring designs according to one or more of the designs shown and described in this patent specification.

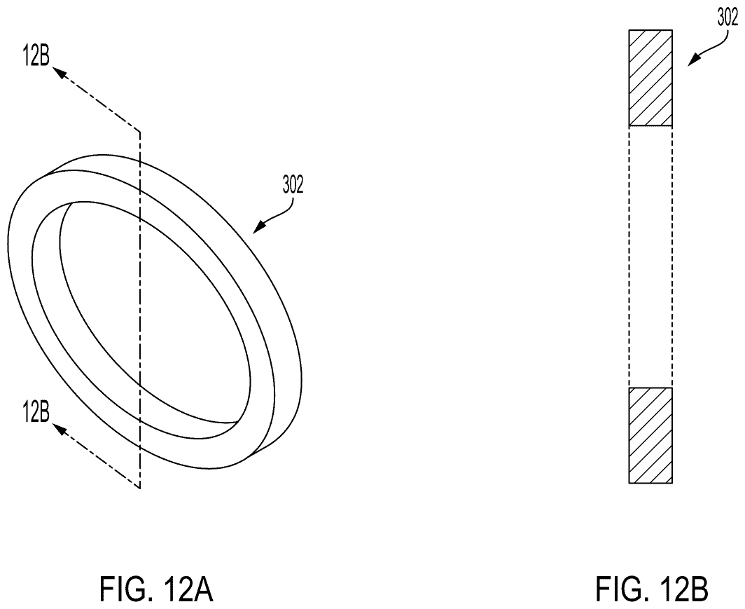

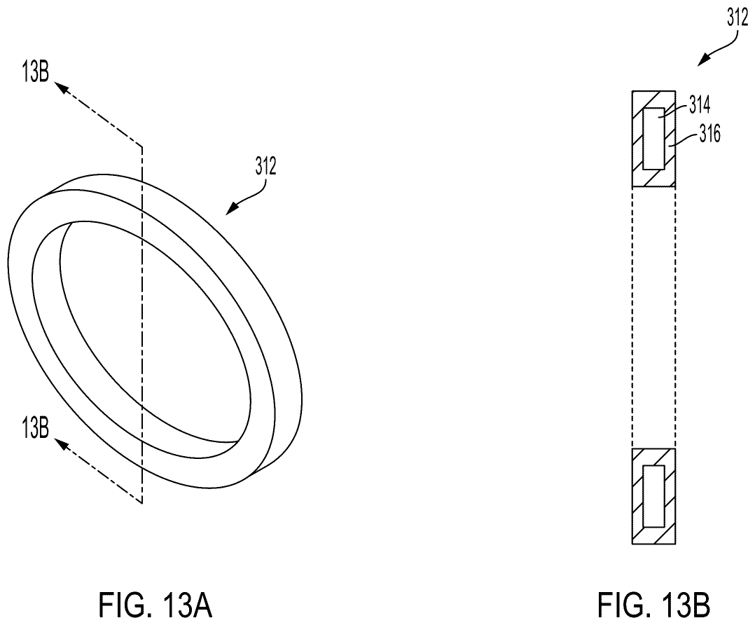

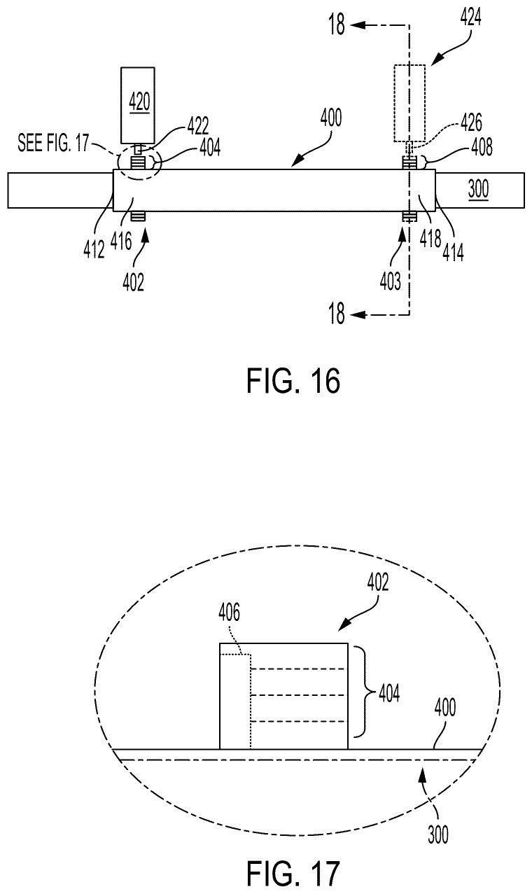

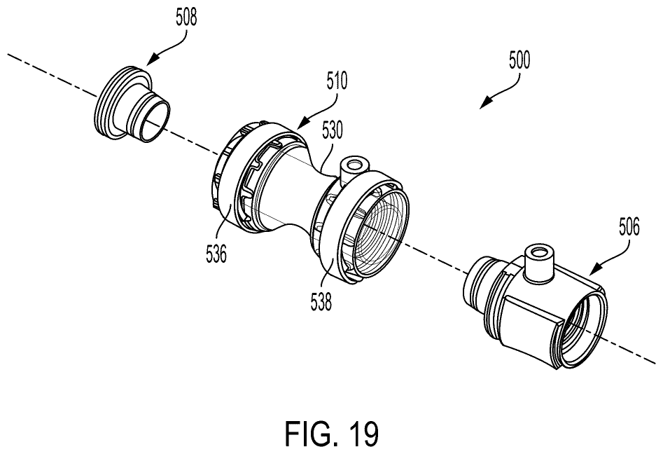

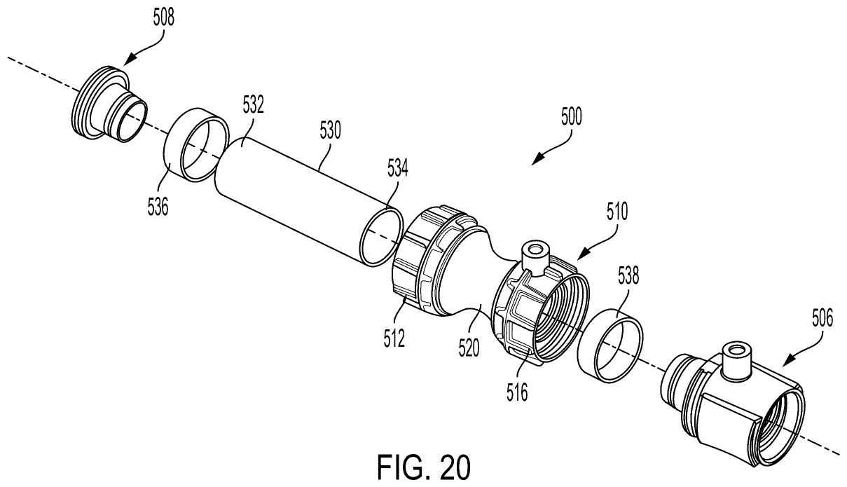

According to some embodiments, the introducer sheath assembly includes an inner tube member with die cut rings, such as that described with reference to FIGS. 6-11. As another example, the inner tube assembly includes an inner tube member with one or more uniform end rings, such as those described with reference to FIGS. 12A-12B. As another example, the inner tube member includes one or more end rings formed of multiple materials, such as those described with reference to FIGS. 13A-13B. As another example, the inner tube member includes one or more end rings formed with a plurality of layers, such as those described with reference to FIG. 16. In various embodiments, the inner tube member secured with one or more clamping members, such as those described with reference to FIG. 19-20.

As shown in FIG. 1, an introducer sheath valve assembly 100 (i.e., a medical device) includes an introducer sheath 102. The introducer sheath 102 may be formed of any material with suitable biocompatible and mechanical properties, for example, fluorinated ethylene propylene (FEP), expanded high density polyethylene (eHDP), or any other material with suitable biocompatible and mechanical properties. The introducer sheath 102 may be of any size including but not limited to from about 12 Fr to 26 Fr or any size therebetween. The introducer sheath valve assembly 100 includes a threaded adapter 104 on an end of introducer sheath 102. The threaded adapter 104 may be formed of any biocompatible plastic or any biocompatible metal with suitable biocompatible and mechanical properties. The threaded adapter 104 may be coupled to the introducer sheath 102 by a variety of means which may include, but is not limited to, adhesives such as polyurethan adhesives, quick setting cyanoacrylate adhesives, or ultraviolet cured adhesives, bonding such as thermal bonding, fusing, mechanical adapters, ultrasonic welding, by an interference fit, insert molding, and so forth.

As shown in FIG. 1, a front fitting 106 (e.g., a housing) may be coupled to the second end of the threaded adapter 104. The front fitting 106 may be designed to include a profile to facilitate a user gripping the introducer sheath valve assembly 100 securely. The front fitting 106 may include one or more protrusions 107 to enhance traction for a user on the introducer sheath valve assembly 100. In some embodiments, the front fitting 106 may be formed with any biocompatible metal or plastic with suitable biocompatible and mechanical properties, and the protrusions 107 may be made of a similar material as the front fitting 106 or may be made of a material with a high coefficient of friction, a material more compliant than the front fitting 106, or made with grating, a roughening, a raised company logo or design, or striations in the surface in conjunction with the material listed above to further aid in the gripping of the device. These features on the surface of the front fitting 106 may also be used to aid in gripping without the use of the protrusions 107 and may be applied directly to a lateral surface (e.g., a gripping or contact surface) of the front fitting 106. In some embodiments, a flush port 109 may extend from the front fitting 106 (e.g., outwardly from a lateral surface of the front fitting 106).

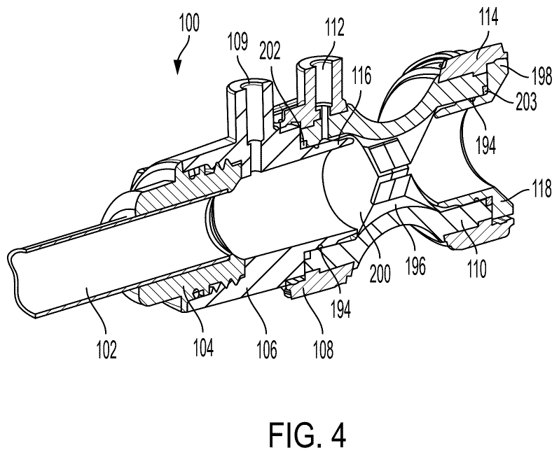

As shown in FIG. 1, an outer tube 110 includes a first collar 108 coupled to the front fitting 106 at a first end of introducer sheath valve assembly 100, a second collar 114 positioned at a second end of the introducer sheath valve assembly 100, and the outer tube 110 coupled between the first collar 108 and the second collar 114. The first collar 108 includes a fill port 112, and the fill port 112 is fluidly coupled with an interior of the outer tube 110. In some embodiments, each of the first collar 108, the second collar 114, and the fill port 112 may be formed of any biocompatible metal or plastic with suitable biocompatible and mechanical properties. Optionally, the fill port 112 may be located at any point along either of the first collar 108 or the second collar 114. As shown in FIG. 4, a rear fitting 198 fits within the outer tube 110 and the second collar 114.

As shown in FIGS. 3-4, the introducer sheath valve assembly 100 includes an inner tube 200 (e.g., a film tube) arranged inside the outer tube 110 and a pressurizable space 196 is defined between the outer tube 110 and the inner tube 200. The inner tube 200 extends along a longitudinal axis and includes a pair of end rings including a first retention feature in the form of a first end ring 202 and a second retention feature in the form of a second end ring 203. As shown in FIG. 4, the inner tube 200 is affixed between the outer tube 110 and the front fitting 106 at the first end ring 202 and between the outer tube 110 and the rear fitting 198 at the second end ring 203.

As shown in FIGS. 3-4, the outer tube 110 includes a pair of sealing lips 194 which may interface with each of the inner tube 200, the front fitting 106, and the rear fitting 198.

In some embodiments, the outer tube 110 may be constructed of any flexible material having desirable mechanical and biocompatible properties, including but not limited to any elastomer, latex, or polycarbonate with desirable mechanical and biocompatible properties. In one embodiment, the outer tube 110 comprises silicone and has an hourglass shape when not pressurized. When the pressurizable space 196 is pressurized to a sufficient pressure such that the inner tube 200 collapses, the outer tube 110 may subsequently distend such that the hourglass shape of the outer tube 110 alters and provides indication of sufficient pressure in the pressurizable space 196 (e.g., when the pressurizable space 196 is pressurized with at least one substance to a sufficient pressure, the inner tube 200 collapses, or distends inwardly around a profile of any object positioned therethrough, for example, a tool or another endoluminal device, which limits back bleeding). This feature of the outer tube 110 facilitates the user of a device to easily and quickly identify the optimal pressure for the device.

FIGS. 3 and 4 illustrate the end rings 202, 203 on either ends of the inner tube 200. That is, end ring 202 is positioned at a first end of inner tube 200 and end ring 203 is positioned at a second end of inner tube 200 opposite the first end. The end rings 202, 203 are used to create a stiff member to aid in attachment of the inner tube 200 to the front fitting 106 and to the rear fitting 198. The end rings 202, 203 may be made of any material with desirable biocompatible and mechanical properties and will be described in greater detail below. The inner tube 200 with the attached end rings 202, 203 may be inserted through the outer tube 110 and attached to the protruding end 116 of the front fitting 106. The first collar 108 with the fill port 112 may then be attached to the front fitting 106 with an adhesive (e.g., adhered), but other suitable attachment methods may suffice. The second end ring 203 may then be attached to the protrusion 118 on the rear fitting 198. The second collar 114 may then be snapped onto the rear fitting 198. That is, the first collar 108 may be coupled to the first end ring 202 and the first collar 108 may be used to mechanically constrain or engage first end ring 202 and the second collar 114 may be coupled to the second end ring 203 and the second collar 114 may be used to mechanically constrain or engage the second end ring 203.

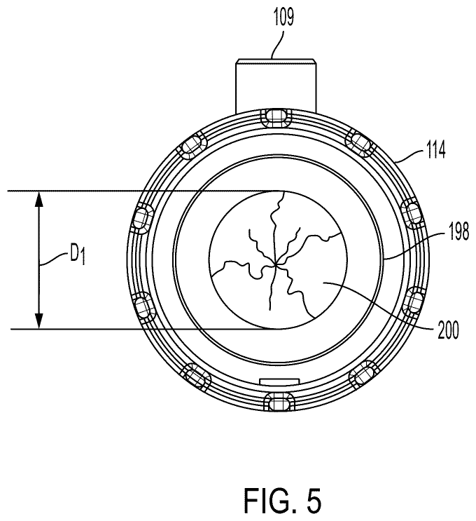

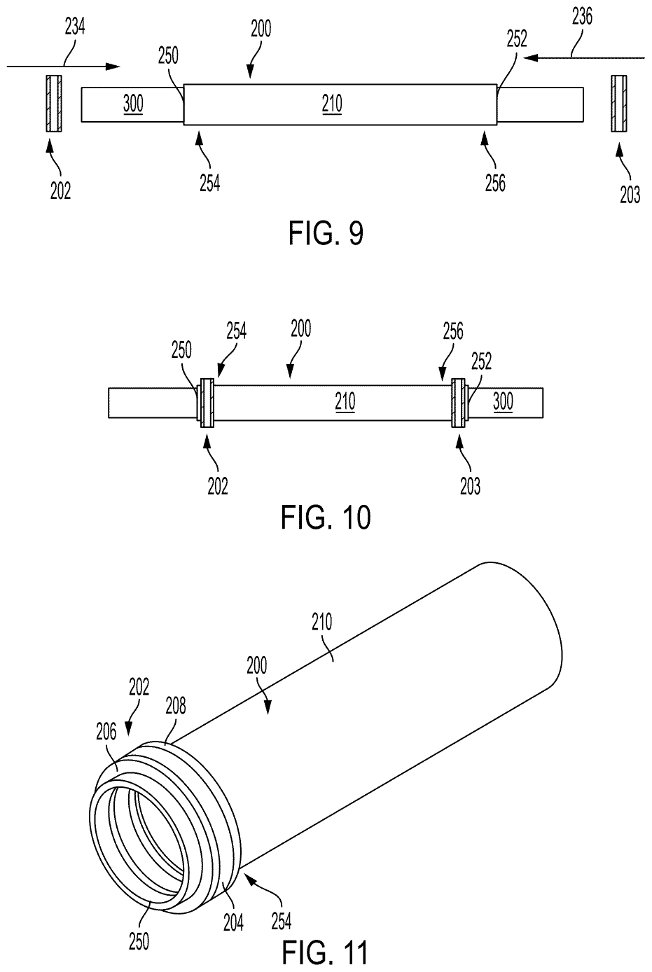

FIG. 5 illustrates an end on view of the device showing a collapsed inner tube 200. The second collar 114 with the fill port 112 (see FIG. 4) has a feature that allows the pressurizable space 196 (see FIG. 4) to be filled to a sufficient pressure to cause the inner tube 200 to collapse around a profile of any object insertable therethrough, for example, a tool or another endoluminal device (e.g., the fill port 112 is fluidly coupled with the pressurizable space 196 to allow fluid to move in (e.g., a fluid input) and out (e.g., a fluid output) of the pressurizable space 196). The pressurizable space 196 may be filled with any suitable material or materials. For example, the pressurizable space 196 may be filled with one or more of the following substances: air, silicone, water, saline solution, low volatility biocompatible liquids, glycerin, propylene glycol, polyethylene glycol, compressible foam, elastomeric spheres, and crosslinked silicone gels. The inner tube 200 has a center portion 210 (see FIG. 9) intermediate the end rings 202, 203 that defines a first diameter D1 that varies with the pressure of the pressurizable space 196. That is, as the pressure within the pressurizable space 196 increases, the diameter D1 decreases such that the inner tube 200 constricts or transitions toward a collapsed configuration. As the inner tube 200 constricts or collapses, the inner tube 200 may contact and conform around a profile of an object positioned therethrough, for example, a tool, to prevent back bleeding through the inner tube 200. As shown in FIG. 9, the inner tube 200 defines a first extent 250, a second extent 252 opposite the first extent, a first portion 254 adjacent the first extent 250, and a second portion 256 adjacent the second extent 252.

As will be described with reference to the design of FIGS. 6-8, the end rings 202, 203 can be constructed of one or more axially adjacent ring portions. As shown in FIG. 6, the end rings 202, 203 include a first ring portion 204, a second ring portion 206, and (in some embodiments) a third ring portion 208 (as shown in Figures, alternatively in some embodiments, not shown in Figures, the end ring may comprise only the first ring portion 204 and the second ring portion 206), where the first ring portion 204 is positioned between, or intermediate to, the second ring portion 206 and the third ring portion 208. That is, each of the first ring portion 204, second ring portion 206, and third ring portion 208 may be positioned axially to each other. As shown in FIG. 6, each of the first ring portion 204, the second ring portion 206, and the third ring portion 208 are axially aligned and longitudinally offset, and the first ring portion 204 is coupled to each of the second ring portion 206 and the third ring portion 208. In some embodiments, the second ring portion 206 is coupled to a first axial side of the first ring portion 204 and the third ring portion 208 is coupled to a second axial side of the first ring portion 204 opposite the second ring portion 206 such that the first ring portion 204 is coupled intermediate the second ring portion 206 and the third ring portion 208.

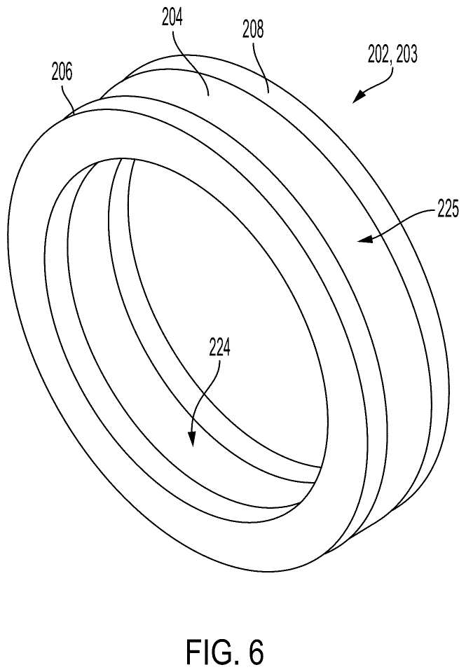

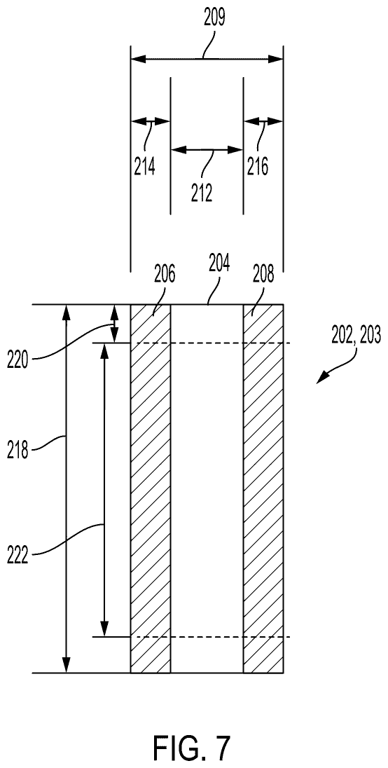

The end rings 202, 203 of the design of FIGS. 6-8 have an axial length 209 which is defined by the combined axial length of each of the second ring portion 206, the first ring portion 204, and the third ring portion 208. The first ring portion 204 defines an axial length 212, the second ring portion 206 defines an axial length 214, and the third ring portion 208 defines an axial length 216. Further, the end rings 202, 203 define an inner surface 224 and an outer surface 225, and end rings 202, 203 define an outer diameter 218, an inner diameter 222, and a radial width 220. In some embodiments, each of the ring portions 204, 206, 208 have the same or substantially the same outer diameters 218, inner diameters 222, and radial widths 220. In some embodiments, each of the second ring portion 206 and the third ring portion 208 have the same axial length 214, 216. In some embodiments, the axial lengths 214, 216 of the second and third ring portions 206, 208 are each less than the axial length 212 of the first ring portion 204. As shown in FIG. 7, the inner diameter 222 can be the same as, or slightly greater than, the diameter D1 (FIG. 5) of the inner tube 200.

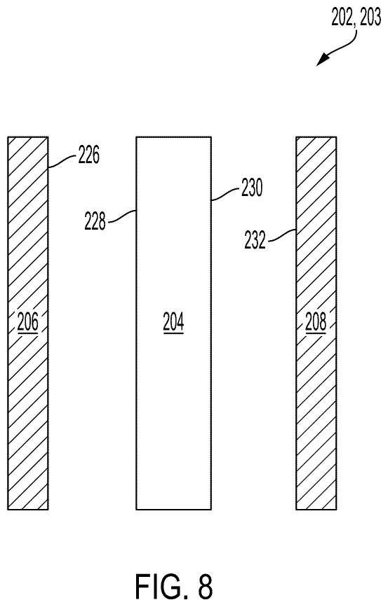

As shown in FIG. 8, the first ring portion 204 defines a first axial face 228 and a second axial face 230 opposite the first axial face 228, the second ring portion 206 defines a first axial face 226 generally facing the first axial face 228 of the first ring portion 204, and the third ring portion 208 defines a first axial face 232 generally facing the second axial face 230 of the first ring portion 204. As shown, the first ring portion 204, the second ring portion 206, and the third ring portion 208 are coupled together to create one or more end ring 202, 203. In some embodiments, an adhesive is present (e.g., coated, imbibed, or otherwise applied) at one or more of the first axial face 228 and the first axial face 226 to couple (e.g., adhere) the second ring portion 206 to the first ring portion 204. Similarly, an adhesive may be present at one or more of second axial face 230 and the first axial face 232 to couple (e.g., adhere) the third ring portion 208 to the first ring portion 204. Additionally or alternatively, the first, second, and/or third ring portions 204, 206, 208, respectively, are bonded (e.g., thermally bonded) to one another.

In embodiments, adhering may include melting or melding one or more materials to join them, applying a topical substance which, when cured, provides holding force between two or more components.

According to the design of FIGS. 6-8, the first ring portion 204 can be formed of a first material, the second ring portion 206 formed of a second material, and the third ring portion 208 of a third material. In some embodiments, the second material is the same as the third material, and the second ring portion 206 is identical to, or similar to, the third ring portion 208. In embodiments, the second material is of the same class of materials as the third material. The first ring portion 204 is optionally formed of a polyethylene material and each of the second ring portion 206 and the third ring portion 208 of an ethyl-vinyl-acetate (EVA) copolymer, for example, although a variety of materials are contemplated. The EVA copolymer can be less than 50% EVA by volume, less than 30% EVA, less than 25% EVA, less than 20% EVA, or less than 10% EVA by volume, for example. In some embodiments, the EVA copolymer is approximately 8% EVA by volume. In some embodiments, the material (e.g., polyethylene) of first ring portion 204 has a structural stiffness greater than (e.g., a higher durometer than) the material (e.g., EVA) of each of the second ring portion 206 and the third ring portion 208. Optionally, the material (e.g., EVA) of each of the second ring portion 206 and the third ring portion 208 is softer than the material (e.g., polyethylene) of the first ring portion 204, and the material (e.g., EVA) of each of the second ring portion 206 and the third ring portion 208 has a lower melting temperature than the material (e.g., polyethylene) of the first ring portion 204.

In the design of FIGS. 6-8, one or both of the second ring portion 206 and the third ring portion 208 may be formed of a low density poly-ethylene (LDPE) material. As alluded to above, in various examples, the first material of the first ring portion 204 may be formed of a structurally stiffer material than each of the second material and third material of the second ring portion 206 and the third ring portion 208, respectively. In various embodiments, the second material of the second ring portion 206 and third material of the third ring portion 208 may be the same material or a different material or may be of the same class of materials. In some examples, the second material and third material (e.g., whether the same or different from each other) are formed of a material with a lower melting point than the first material of the first ring portion 204. In some embodiments, the first material of the first ring portion 204 is the same material as the inner tube 200 (e.g., expanded polyethylene). In some embodiments, the first material of the first ring portion 204 is of the same type or same class of polymer as the inner tube 200 (e.g., a polyethylene).

In some embodiments, the end rings 202, 203 are formed (e.g., die-cut) by joining together two or more layers of material. That is, a first layer formed of the first material is laid down, a second layer formed of the second material is laid down on the first layer, and a third layer formed of the third material is laid down on the second layer, such that the layers of the first layer, second layer, and third layer are defined. The layers may be coupled together via an adhesive, bonding agent, sonic welding, heat welding, or otherwise coupled to define a layered construct. The layers may be coupled in the presence or absence of pressure, and may be laminated, for example. The end rings 202, 203 may ultimately be formed (e.g., die cut) from the layered construct. Additional forming processes may be incorporated. The end rings 202, 203 may be post-processed to ensure consistent physical characteristics. For example, the end rings 202, 203 may have any of a variety of surface treatments applied (e.g., including machining or sanding) to smooth out one or more surfaces of the end ring 202, 203).

In some embodiments, the end rings 202, 203 are formed by first constructing the individual ring layers by one or more methods including cutting, stamping, extruding, printing, or the like. The constituent layers of the end rings 202, 203 may be subsequently laminated (e.g., heat welded) and pressed together to form the end rings 202, 203. That is, each layer of end rings 202, 203 may be a ring portion (e.g., first ring portion 204, second ring portion 206, third ring portion 208, etc.). The end rings 202, 203 may be machined (e.g., sanded) to smooth out one or more surfaces of the end ring 202, 203. In some embodiments, the end rings 202, 203 may be otherwise post-processed to ensure consistent physical characteristics of the end rings.

Inner tube 200 may be constructed of any very thin, strong, drapeable material such as expanded polyethylene (ePE), expanded polytetrafluoroethylene (ePTFE), FEP, Aliphatic Thermoplastic Polyether Polyurethane (ATPU), fabrics, silk, or Kevlar® brand fiber, and combinations thereof. These materials may be used as a single layer construct or a multi-layer construct, and may be formed as composite materials. For example, the inner tube 200 may be formed of a thin, porous polymeric substrate including multiple layers of material, which may be filled or imbibed with a secondary polymer. The secondary, filling or imbibing polymer may be the same as or similar to that of the construct or may be a different polymer.

As illustrated in FIG. 9, the inner tube 200 may be formed, or extruded (e.g., by an extruder and/or in an extruding process) on a mandrel 300. The mandrel 300 may be formed of a suitable metal, a coated material, or other suitable material. In various examples, the inner tube 200 is extruded, wrapped, or otherwise formed onto the mandrel 300 and subsequently removed. As previously referenced, the inner tube 200 may be a film tube formed of a thin, strong, flexible, and drapeable material (e.g., a compliant material), and in the case of mandrel formation that material will generally be capable of being formed onto the mandrel 300 (e.g., in a flowable state or a solid state). In various examples, the material is malleable during formation of the inner tube 200 and is capable of being formed around, and on the mandrel 300. Following formation, or both following and before formation, the inner tube 200 may be generally compliant in a solid state. The compliant material may be a material capable of elastically deforming and collapsing around or draping around-and creating a seal around-a tool inserted through the introducer sheath valve assembly 100 under an increased pressure within the pressurizable space 196. The compliant material may be an inelastic material capable of deforming and collapsing around or draping around-and creating a seal around-a tool inserted through the introducer sheath valve assembly 100 under an increased pressure within the pressurizable space 196.

Regardless of the specific formation method, the inner tube 200 may be formed of a variety of compliant materials, including any of those previously described, which have suitable biocompatible and mechanical properties for the desired application (e.g., expanded polyethylene (ePE)).

The inner tube 200 may be compliant and deformable within the introducer sheath valve assembly 100 when pressurized to allow for proper sealing (to itself and to a device inserted through the valve) to prevent back bleeding. Optionally, the inner tube 200 may be formed of a polyethylene material (e.g., ePE) due to availability, cost, environmental factors, production, and strength. Additional material(s) may be employed with ePE, or other materials for the inner tube 200, to reduce or minimize friction. For example, some embodiments include use of an ePE with a surface treatment on an inward-facing surface of the inner tube 200 to decrease the coefficient of friction between the inner tube 200 and the mandrel 300 during manufacture and/or reduce friction with devices that are inserted through the inner tube 200 in use.

As shown in FIGS. 9-10, the first end ring 202 may be positioned onto the inner tube 200 by moving the first end ring 202 onto inner tube 200 (e.g., in a direction 234), over the first extent 250 adjacent the first portion 254 (e.g., first end of inner tube 200). The second end ring 203 may be positioned onto the inner tube 200 by moving the second end ring 203 onto the inner tube 200 (e.g., in a direction 236), over the first portion 254 adjacent the second portion 256 (e.g., second end of inner tube 200 opposite of the first end).

As shown in FIGS. 10-11, the end rings 202, 203 may be coupled to the inner tube 200. In some embodiments, the end rings 202, 203 may be coupled to the inner tube 200 by reflowing, or melting, the second ring portion 206 and the third ring portion 208 of the end rings 202, 203 to adhere or bond the end rings 202, 203 to inner tube 200. In some embodiments, end rings 202, 203 may be positioned on the inner tube 200 (e.g., on the first portion 254 and the second portion 256, respectively). The end rings 202, 203 may then be heated to a processing temperature greater than the melting point of the second material of the second ring portion 206 and third material of the third ring portion 208 and less than the melting point of the first material of the first ring portion 204 and the inner tube 200. That is, the end rings 202, 203 and the inner tube 200 may be heated to the processing temperature and the second ring portion 206 and the third ring portion 208 of the first end ring 202 and the second end ring 203 are reflowed, or melted, to adhere the end rings 202, 203 to the inner tube 200. Optionally, the material of the second ring portion 206 and the third ring portion 208 (e.g., EVA) has a melting temperature point of lower than 130 degrees Celsius (° C.), and in some embodiments has a melting temperature point of approximately 100 to 110 degrees Celsius (° C.). In embodiments, the material of the second ring portion 206 and the third ring portion 208 (e.g., EVA) has a melting temperature point equal to 130 degrees Celsius (° C.). In some embodiments, the material of the first ring portion 204 and the inner tube 200 (e.g., polyethylene or expanded polyethylene) has a melting point of less than 135 degrees Celsius (° C.), and optionally has a melting point of approximately 125 to 135 degrees Celsius (° C.). Materials with lower melting points allows for the processing temperature to be lower which is easier to achieve and maintain during a manufacturing process, and a lower processing temperature maintains and/or reduces the degradation of the molecular structure of the inner tube 200 or portions of the end rings 202, 203.

FIGS. 12A-13B illustrate several embodiments of additional end ring designs and features that may be implemented as an alternative to or in addition to the end ring concepts and features described above. For example, FIGS. 12A-12B show an end ring 302 have features that may be utilized for one or both of the end rings 202, 203. The end ring 302 is comprised of a uniform material with suitable biocompatible and mechanical properties. In some embodiments, the end ring 302 is comprised of a first material which has a lower melting point than the material of the inner tube 200. Optionally, the end ring 302 is formed of a uniform EVA copolymer. Optionally, the end ring 302 is formed of an LDPE material. Optionally, the end ring 302 is assembled with the inner tube 200 in substantially the same manner as the end rings 202, 203. That is, the end ring 302 may be placed on the inner tube 200 on the first portion 254 adjacent the first extent 250 and may be subjected to a processing temperature to melt, or flow, the material of the end ring 302 to adhere end ring 302 to the inner tube 200.

FIGS. 13A-13B show another end ring 312 with features that may be utilized for one or both of the end rings 202, 203. The end ring 312 is comprised of a first portion 314 formed of a first material, and a second portion 316 that surrounds the first portion 314, and the second portion 316 is formed of a second material. In some embodiments, one or both of the first material of the first portion 314 and the second material of the second portion 316 is formed of suitable biocompatible and mechanical properties. In some embodiments, the first material of the first portion 314 is a fibrous material, a metallic material, or another structurally rigid material. Optionally, the second material of the second portion 316 is formed of an ethyl-vinyl-acetate (EVA). In some embodiments, the second portion 316 is formed of a LDPE material. In some embodiments, the second portion 316 of the end ring 302 is comprised of a first material which has a lower melting point than the material of the inner tube 200. Optionally, the end ring 302 is formed into a single uniform ring and is formed of a uniform EVA copolymer. In some embodiments, the end ring 302 is assembled with the inner tube 200 in substantially the same manner as the end rings 202, 203. That is, the end ring 302 may be placed on the inner tube 200 on the first portion 254 adjacent the first extent 250 and may be subjected to a processing temperature to melt, or flow, the second material of the second portion 316 of the end ring 302 to adhere the end ring 302 to the inner tube 200. That is, the first portion 314 may be the structural component of the end ring 302 and the second portion 316 may be melted or flowed to adhere the end ring 302 to the inner tube 200.

In some embodiments, each of the end rings 202, 302, and 312 may have the same, or similar dimensions. In some embodiments, each of the end rings 202, 302, 312 may have the same, or similar dimensions before the assembly of the end rings 202, 302, 312 on the inner tube 200 and the end rings 202, 302, 312 may subsequently have different dimensions after coupling to the inner tube 200. In some embodiments, each of the end rings 202, 302, 312 may have different dimensions before the assembly of the end rings 202, 302, 312 on the inner tube 200 and the end rings 202, 302, 312 may subsequently have the same, or similar dimensions after coupling to the inner tube 200.

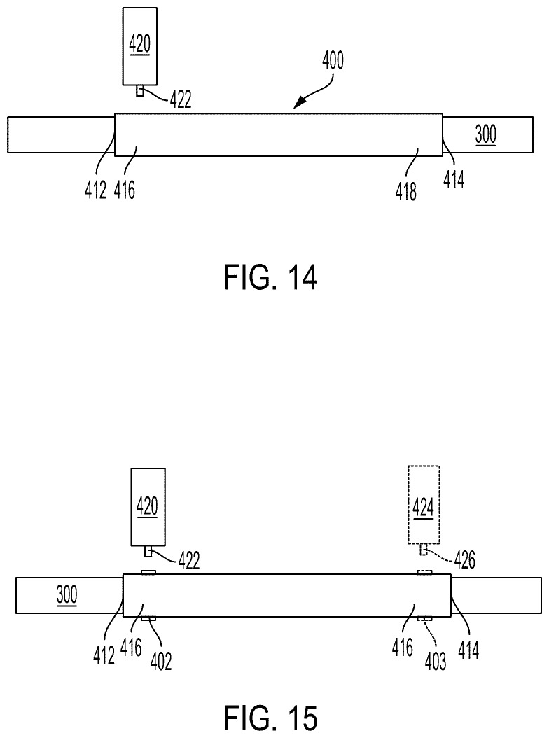

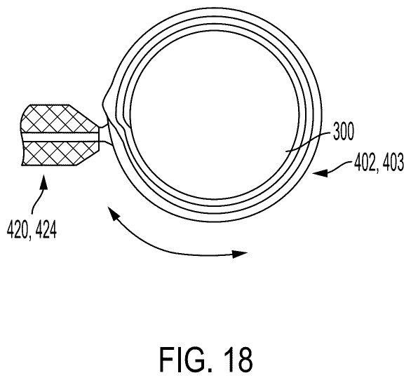

FIGS. 14-18 show another inner tube 400 that may be formed similarly to the inner tube 200. The inner tube 400 may be extruded onto the mandrel 300 to define a length between a first extent 412 and a second extent 414. The inner tube 400 includes a first portion 416 adjacent the first extent 412 and a second portion 418 adjacent the second extent 414. The inner tube 400 may be assembled with a pair of end rings 402, 403 (e.g., a first end ring 402 and a second end ring 403; FIGS. 15-18) which may include substantially similar properties with respect to the end rings 202, 302, or 312 but may be formed and coupled to the inner tube 400 in a different manner.

As shown in FIG. 14, an extruder 420 may include a nozzle 422, and the extruder 420 may be positioned adjacent the mandrel 300, and the extruder 420 may be coupled to an electronic controller which may control an extrusion rate, movement of the extruder 420, temperature of the extrusion, and other characteristics of the extruder 420. The extruder 420 may also be coupled to a hopper configured to hold material. The extruder 420 may be positioned to extrude material onto the inner tube 400 to create the pair of end rings 402, 403 (FIG. 15). Optionally, the extruder 420 is capable of additive manufacturing, or 3D printing. That is, the extruder 420 melts, or flows, material from the hopper, through the nozzle 422, and onto the workpiece (e.g., the inner tube 400), and the end rings 402, 403 are formed as the material cools and/or cures.

As shown in FIG. 15, the extruder 420 flows material onto the first portion 416 of the inner tube 400 while the mandrel 300 rotates, allowing the extruder 420 to lay a material along the circumference of the inner tube 400 at a discrete axial position (e.g., along the first portion 416 adjacent the first extent 412). The mandrel 300 may rotate a discrete number of rotations to create an extruded end ring 402, 403 around the inner tube 400 with a plurality of radially adjacent layers 404 (FIG. 16). Optionally, a keyed portion 406 is formed within the end ring 402, 403 (see FIG. 17). In some embodiments, the keyed portion 406 is formed during the additive manufacturing process. In some embodiments, the keyed portion 406 is formed by a material removal manufacturing process. In some embodiments, the keyed portion 406 is a recessed portion, or female portion within the end ring 402, 403, and one or more of the first collar 108, the second collar 114, the front fitting 106, and the rear fitting 198 includes a corresponding keyed feature (not shown) to fit with or interface with the keyed portion 406. Optionally, one or more of the first collar 108, the second collar 114, the front fitting 106, and the rear fitting 198 includes a protruding portion, or male portion to fit in the keyed portion 406. It is understood that the reverse may be true such that the end rings 402, 403 may include the male portion of a keyed feature and one or more of the first collar 108, the second collar 114, the front fitting 106, and the rear fitting 198 may include the female portion.

In some embodiments, the extruder 420 extrudes a material that has suitable biocompatible and mechanical properties such as ePE, polyethylene, LDPE, ePTFE, PTFE, EVA, FEP, or another type of polymer suitable for extrusion from extruder 420.

As shown in FIGS. 16-18, the plurality of layers 404 may be formed in various ways (e.g., based upon extrusion rate, number of layers, etc.) to create different layer thicknesses or different layer widths. Each of the material, the layer thickness, layer width, and number of layers may be varied to alter mechanical characteristics of the end rings 402, including mechanical characteristics such as the rigidity, strength, stiffness, elasticity, or other mechanical characteristics.

As shown in FIGS. 16-18, an extruder 424 includes a nozzle 426 which may form the end ring 403 while the extruder 420 forms the end ring 402. That is, the extruder 424 may form the plurality of layers 408 of the end ring 403 as the mandrel 300 rotates. In some embodiments, the end rings 402, 403 are formed by only one extruder 420. In some embodiments, the end rings 402, 403 are formed by two extruders 420, 424.

The end rings 402, 403 may be used in substantially the same manner as the end rings 202, 203. That is, the inner tube 400 may replace the inner tube 200 in the introducer sheath valve assembly 100. That is, the end rings 402, 403 are used to create a stiff member to aid in attachment of the inner tube 400 to the front fitting 106 and to the rear fitting 198. The end rings 402, 403 may be made of any material with desirable biocompatible and mechanical properties. The inner tube 400 with the end ring 402, may be inserted through the outer tube 110 and attached to the protruding end of the front fitting 106. The first collar 108 with the fill port 112 may then be attached to the front fitting 106 with an adhesive, but other suitable attachment methods may suffice. The second end ring 403 may then be attached to the protrusion on the rear fitting 198. The second collar 114 may then be snapped onto the rear fitting 198. That is, the first collar 108 may be used to mechanically constrain or engage the first end ring 402 and the second collar 114 may be used to mechanically constrain or engage the second end ring 403.

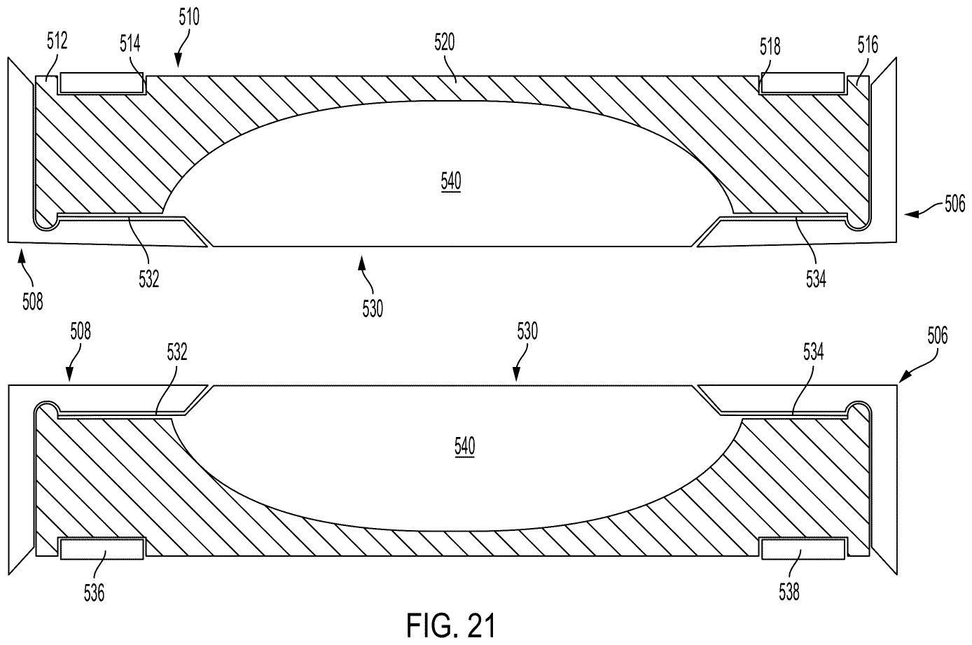

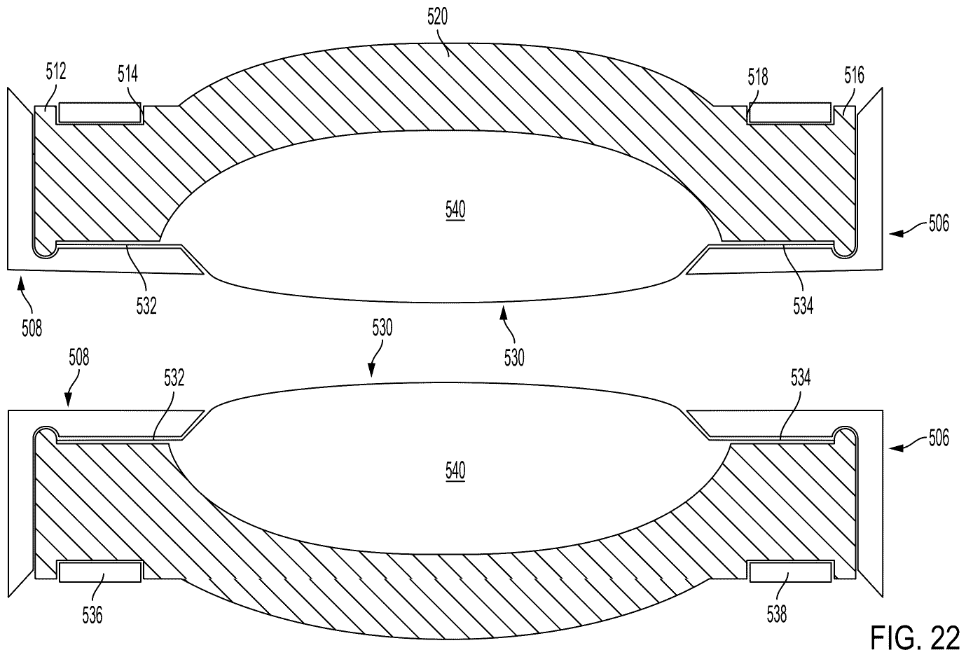

As shown in FIGS. 19-22, an introducer sheath assembly 500 is provided which may be similar to the introducer sheath valve assembly 100 (FIG. 1). The introducer sheath assembly 500 may include a front fitting 506 (similar to the front fitting 106), a rear fitting 508 (similar to the rear fitting 198), an outer tube 510, and an inner tube 530. Outer tube 510 surrounds the inner tube 530 to create a pressurizable space 540. In some embodiments, a fill port (not shown, similar to the fill port 112) is fluidly coupled to the pressurizable space 540 which may be filled with an appropriate fluid or substance. For example, the pressurizable space 540 may be filled with one or more of the following substances: air, silicone, water, saline solution, low volatility biocompatible liquids, glycerin, propylene glycol, polyethylene glycol, compressible foam, elastomeric spheres, and crosslinked silicone gels.

As shown in FIGS. 19-22, the outer tube 510 includes a first end portion 512 and a second end portion 516, and the first end portion 512 defines a first recess 514 and the second end portion 516 defines a second recess 518. In some embodiments, the outer tube 510 is generally cylindrical and each of the first recess 514 and the second recess 518 are circumferential around the entirety of the first end portion 512 and the second end portion 516, respectively. Optionally, each of the first recess 514 and the second recess 518 surround only a portion of the circumference of each of the first end portion 512 and the second end portion 516, respectively. Further, outer tube 510 defines a center portion 520 longitudinally intermediate the first end portion 512 and the second end portion 516.

As shown in FIGS. 19-22, the inner tube 530 of the introducer sheath assembly 500 includes a first end portion 532 and a second end portion 534 opposite the first end portion 532. In some embodiments, the first end portion 532 of the inner tube 530 is positioned radially intermediate the rear fitting 508 and the first end portion 512 of the outer tube 510 and the second end portion 534 of the inner tube 530 is positioned radially intermediate the front fitting 506 and the second end portion 516 of the outer tube 510.

The introducer sheath assembly 500 also includes a first clamping member 536 and a second clamping member 538. Illustratively, the first clamping member 536 is positioned within the first recess 514 and the second clamping member 538 is positioned within the second recess 518. The clamping members 536, 538 may be a full ring (e.g., surround the circumference of the outer tube 510), or may be a partial ring (e.g., surround a portion of the circumference of the outer tube 510). In some embodiments, the clamping members 536, 538 may be sized and shaped to fill the recesses 514, 518, respectively. The clamping member 536 surrounds and compresses each of the first end portion 512 of the outer tube 510 and the first end portion 532 of the inner tube 530 to the rear fitting 508 and the clamping member 538 surrounds and compresses each of the second end portion 516 of the outer tube 510 and the second end portion 534 of the inner tube 530 to the front fitting 506. That is, the clamping members 536, 538 mechanically constrain or engage the outer tube 510 and the inner tube 530 to each of the front fitting 506 and the rear fitting 508.

The outer tube 510 may be an hourglass shaped flexible member (similar to the outer tube 110) which may be capable of distending under pressure. The outer tube 510 may be constructed of any elastomer, latex or polycarbonate with desirable mechanical and biocompatible properties. In one embodiment, the outer tube 510 comprises silicone and has an hourglass shape when not pressurized. When pressurized, the hourglass shape of the outer tube 510 becomes distended to indicate a desirable pressure in the pressurizable space 540 (e.g., when the pressurizable space 540 is pressurized with at least one substance to a sufficient pressure to cause the inner tube 530 to collapse such that the pressure is sufficient to prevent back bleeding). This feature of the outer tube 510 facilitates the user of a device to easily and quickly identify the optimal pressure for the device. Further, the inner tube 530 may also be formed of a compliant and conformable material such as an ePE, LDPE, FEP, ePTFE, or another type of polymer.

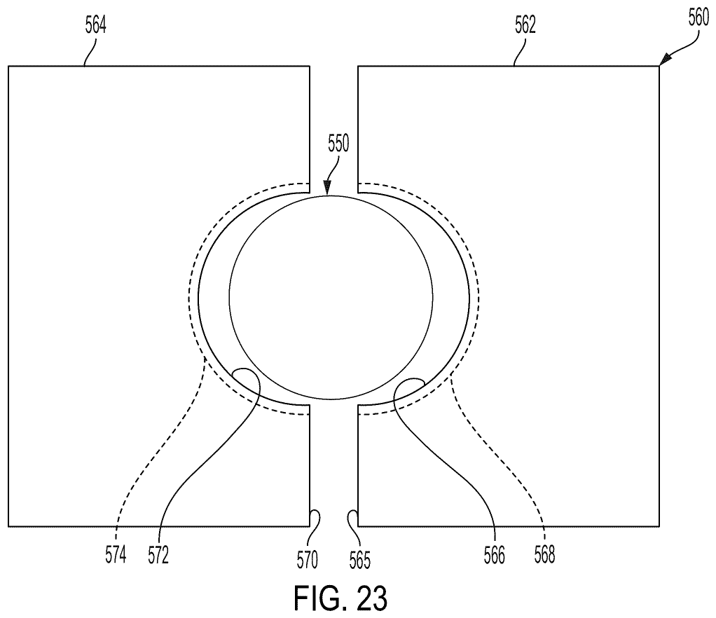

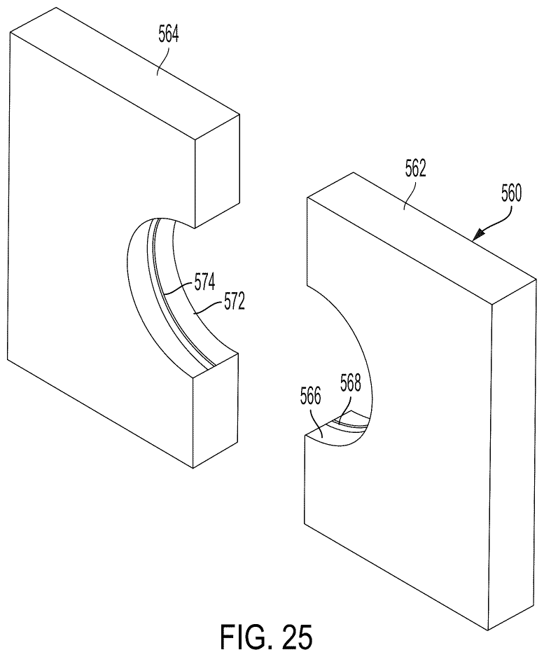

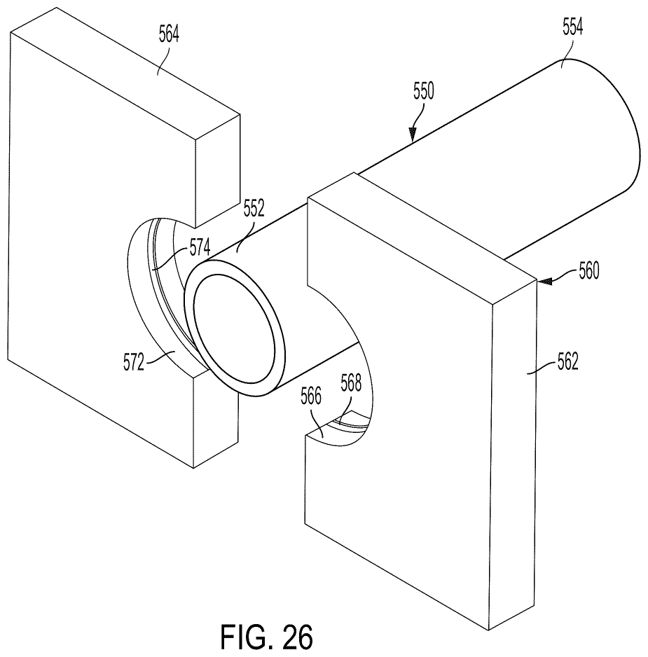

As shown in FIGS. 23-27, an inner tube 550 may be formed by a molding apparatus 560. Molding apparatus 560 may include a first portion 562 and a second portion 564. Optionally, the first portion 562 defines an inner face 565 comprising a mold face 566. In some embodiments, the mold face 566 is generally arcuate, or circular, and is dimensioned to fit around an outer surface of the inner tube 550 and the mold face 566 defines a mold recess 568. In some embodiments, the second portion 564 defines an inner face 570 comprising a mold face 572 and the mold face 572 is generally arcuate, or circular, and is dimensioned to fit around an outer surface of the inner tube 550, and the mold face 572 defines a mold recess 574.

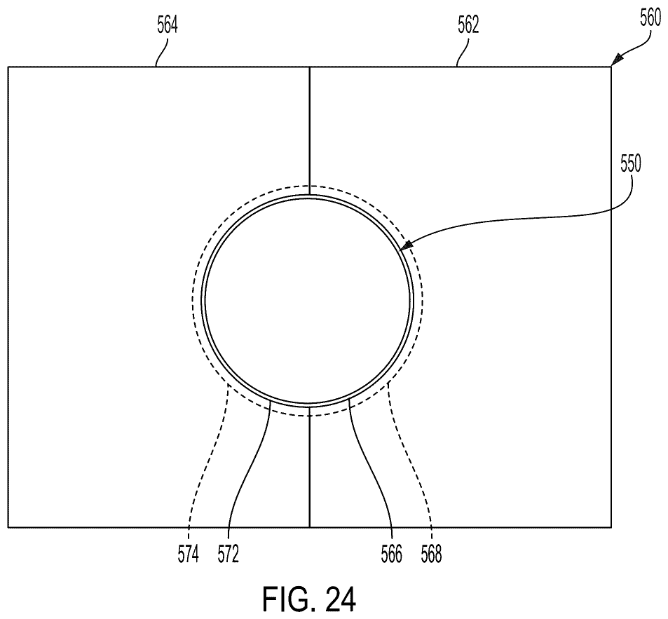

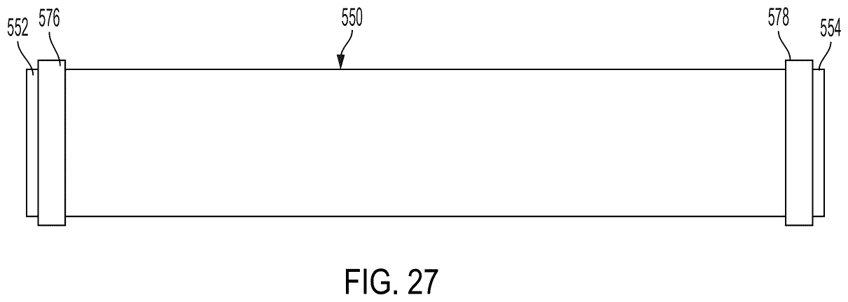

As shown in FIGS. 23-24, the inner tube 550 defines a first end 552 and a second end 554 opposite the first end 552. The first end 552 of the inner tube 550 may be placed between the first portion 562 and the second portion 564 of the molding apparatus 560. The molding apparatus 560 may be operable to close around the first end 552 of the inner tube 550 and seal the recesses 568, 574 around the outer surface of the inner tube 550. The molding apparatus 560 may also be operable to inject a flowing material (e.g., LDPE or EVA) into the recesses 568, 574 to create a first end ring 576 (FIG. 27). Similarly, the second end 554 of the inner tube 550 may be placed between the first portion 562 and the second portion 564 of the molding apparatus 560. The molding apparatus 560 may be operable to close around the second end 554 of the inner tube 550 and seal the recesses 568, 574 around the outer surface of the inner tube 550. The molding apparatus 560 may also be operable to inject a flowing material (e.g., LDPE or EVA) into the recesses 568, 574 to create a second end ring 578 (FIG. 27).

In some embodiments, an end ring (e.g., a uniform material end ring such as end ring 576, a multi-material end ring such as end ring 202, 302, 312) may be formed or coupled onto an inner tube (e.g., the inner tube 200) by one or more coupling processes. In some embodiments, an end ring (e.g., end ring 202, 302, 312) may be coupled to an inner tube (e.g., inner tube 200) by a welding process. An end ring (e.g., end ring 202, 302, 312) may be coupled to an inner tube (e.g., inner tube 200) by an ultrasonic welding process. An end ring (e.g., end ring 202, 302, 312) may be coupled to an inner tube (e.g., inner tube 200) by a spin-welding process.

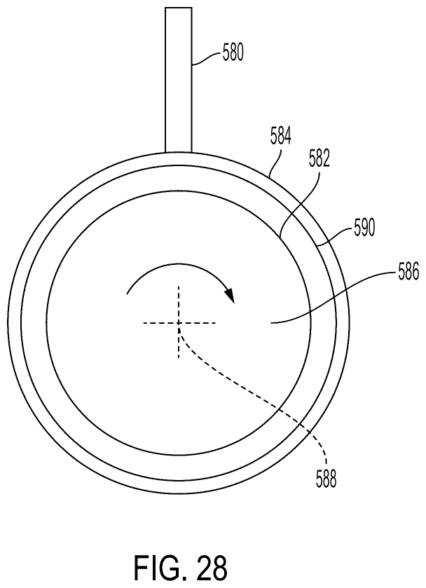

As shown in FIG. 28, an end ring may be coupled with an inner tube by an ultrasonic welding process. In some embodiments, an end ring 584 formed of a thermoplastic material may be placed on an inner tube 582 formed of a thermoplastic material. The inner tube 582 and the end ring 584 may be placed on a mandrel 586 and rotated about an axis 588. A horn, or contact member 580, may be placed radially outwardly from the inner tube 582 and the end ring 584 and operable to clamp the inner tube 582 and the end ring 584 together. The contact member 580 may be operable to vibrate at high frequencies (e.g., 20 KHZ- 40 kHz) to impart frictional heat between the inner tube 582 and the end ring 584. The frictional heat between inner tube 582 and the end ring 584 may melt, or flow, each of the inner tube 582 and the end ring 584 at a contact interface 590 between the inner tube 582 and the end ring 584. Each of the inner tube 582 and the end ring 584 may be melted together and then solidified to join or couple the inner tube 582 and the end ring 584. In some embodiments, a second end ring (not shown) on a second end (not shown) of the inner tube 582 may be formed by the ultrasonic welding process.

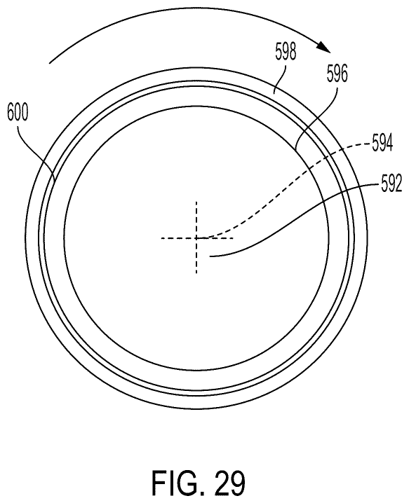

As shown in FIG. 29, an end ring may be coupled with an inner tube by a spin welding process. An inner tube 596 formed of a thermoplastic material may be placed on a mandrel 592 which extends along an axis 594. An end ring 598 formed of a thermoplastic material which may be oversized relative to the inner tube 596 may be placed on the inner tube 596. The end ring 598 may be rotated at high speeds relative to the inner tube 596. The end ring 598 may be decelerated and compressed into the inner tube 596 at a contact interface 600. As the end ring 598 rotates on the inner tube 596 at contact interface 600, frictional heat is created to flow, or melt the end ring 598 and the inner tube 596. The end ring 598 and the inner tube 596 are flowed or melted at the contact interface 600 and the inner tube 596 are coupled together as the flowed material is solidified together to couple the end ring 598 and the inner tube 596 together. In some embodiments, a second end ring (not shown) on a second end (not shown) of the inner tube 596 may be formed by the spin welding process.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

What is claimed is:

1. An introducer sheath assembly, comprising:
an outer tube;
an inner tube arranged at least partially within the outer tube to create a pressurizable space between the outer tube and the inner tube, the inner tube formed of a drapeable material and comprising a first end, a second end, a first end ring adjacent the first end, the first end ring comprising a first ring portion formed of a first material and a second ring portion formed of a second material and coupled to the first ring portion.

2. The introducer sheath assembly of claim 1, further comprising a fitting coupled to the outer tube relative to the inner tube to define the pressurizable space, the fitting operable to engage the inner tube at the first end ring.

3. The introducer sheath assembly of claim 1, further comprising a second end ring adjacent the second end, the second end ring comprising a first ring portion formed of the second material and a second ring portion coupled to the first ring portion.

4. The introducer sheath assembly of claim 3, further comprising a collar operable to engage the second end ring.

5. The introducer sheath assembly of claim 1, wherein the inner tube is formed of a third material, and the second material has a lower melting point than the third material.

6. The introducer sheath assembly of claim 5, wherein the third material is the same as the first material.

7. The introducer sheath assembly of claim 1, wherein the first ring portion and the second ring portion are axially offset.

8. The introducer sheath assembly of claim 1, wherein the outer tube is formed of a flexible material such that upon pressurizing the pressurizable space, the outer tube distends outwardly and the inner tube is formed of a flexible material such that upon pressurizing the pressurizable space, the inner tube distends inwardly.

9. The introducer sheath assembly of claim 1, wherein the inner tube is formed of one of an expanded polyethylene (ePE) and an Aliphatic Thermoplastic Polyether Polyurethane (ATPU).

10. A method of utilizing an introducer sheath assembly including an outer tube, an inner tube formed of a drapeable material, the inner tube arranged within the outer tube and including a first end, a second end, a first end ring adjacent the first end, a second end ring adjacent the second end ring, a center portion between the first end ring and the second end ring, a pressurizable space defined between the inner tube and the outer tube, the first end ring comprising a first ring portion formed of a second material and a second ring portion formed of a third material, the first ring portion coupled to the second ring portion, a first collar coupled to the outer tube relative to the inner tube such that the first end ring is engaged by the first collar, and a fill port fluidly coupled to the pressurizable space, the method comprising:
providing a tool insertable into the inner tube;
pressurizing the pressurizable space with a fluid input to the fill port; and
reducing a diameter of the center portion in response to the pressurizing such that the center portion sealingly collapses around the tool.

11. The method of claim 10, wherein the outer tube is formed of a flexible material, the method further comprising:
distending the outer tube in response to the pressurizing.

12. The method of claim 10, wherein the second end ring comprises a first ring portion formed of the second material and a second ring portion formed of the third material.

13. The method of claim 10, further comprising a second collar coupled to the outer tube relative to the inner tube such that the second end ring is engaged by the second collar.

14. The method of claim 10, wherein the second material has a lower melting point than the third material.

15. The method of claim 14, wherein the first ring portion with the second material is adhered with the first material to couple the first ring portion to the inner tube.

16. The method of claim 10, wherein the inner tube is formed of one of an expanded polyethylene (ePE) and an Aliphatic Thermoplastic Polyether Polyurethane (ATPU).

17. A method of manufacturing a medical device comprising:
forming an inner tube formed of a drapeable material and defining a first end portion, a second end portion, and a center portion, wherein the inner tube is formed of a first material;
forming a first end ring defining a first ring portion formed of a second material and a second ring portion coupled to the first ring portion, the second ring portion formed of a third material different from the second material;
positioning the first end ring adjacent the first end portion of the inner tube; and
coupling the first end ring to the inner tube by adhering the first ring portion to the inner tube.

18. The method of claim 17, further comprising:
forming a second end ring defining a first ring portion formed of the second material and a second ring portion coupled to the first ring portion, the second ring portion formed of the third material.

19. The method of claim 17, wherein the second material has a melting temperature less than the third material.

20. The method of claim 17, wherein the inner tube is formed of one of an expanded polyethylene (ePE) and an Aliphatic Thermoplastic Polyether Polyurethane (ATPU).
